(19) United States
(12) Patent Application Publication
(10) Pub. No.: US 2018/0078140 A1
(43) Pub. Date: Mar. 22, 2018

(54) BIOLOGICAL SIGNAL DETECTION DEVICE

(71) Applicant: KABUSHIKI KAISHA TOSHIBA, Tokyo (JP)

(72) Inventor: Ikkan MIKAMI, Inagi Tokyo (JP)

(21) Appl. No.: 15/448,606

(22) Filed: Mar. 3, 2017

(30) Foreign Application Priority Data
Sep. 16, 2016 (JP) 2016-181405

(51) Int. Cl. A61B 5/00; G01J 1/18; A61B 5/021; A61B 5/1455

(52) U.S. Cl. CPC A61B 5/0059; G01J 1/18; A61B 5/14552; A61B 5/02108; A61B 5/4884; A61B 5/7225

(57) ABSTRACT

A biological signal detection device includes a reference pulse generation unit, a photo-reflector, a reference voltage control unit, a variation amount detection unit, and a biological signal generation unit. The reference pulse generation unit generates a reference pulse train having a variable frequency. The photo-reflector generates a photo-reflector output signal train in response to the reference pulse train. The reference voltage control unit provides a reference voltage. The variation amount detection unit receives the reference voltage and the photo-reflector signal train and generates a variation amount detection signal train. The variation amount detection signal train is provided to the biological signal generation unit to correct the frequency of the reference signal pulse train and to the reference voltage control unit to adjust the reference voltage signal. Adjusting the frequency of the reference pulse signal train improves sensitivity and adjusting the reference voltage signal maintains accuracy of the device.

### CROSS-REFERENCE TO RELATED APPLICATION

This application is based upon and claims the benefit of priority from Japanese Patent Application No. 2016-181405, filed Sep. 16, 2016, the entire contents of which are incorporated herein by reference.

### FIELD

Embodiments described herein relate generally to a biological signal detection device.

### BACKGROUND

In recent years, biological signal detection devices that detect biological information as an object to be measured have been developed in various areas. The biological signal detection devices output a biological detection signal using an analog front-end circuit that performs signal processing on a sensor output signal that is output from a sensor unit.

In the biological signal detection device, pulse wave measurements of a living body and stress measurements, SpO2 (percutaneous arterial blood oxygen saturation) measurements, blood pressure estimation, or the like using the pulse wave are calculated using the photo-reflector or the like as a sensor unit.

A biological detection signal observed by the biological signal detection device has a weak signal level and is a signal whose S/N ratio is very small. For this reason, it is difficult to calculate pulse wave measurements, stress measurements, SpO2 measurements, blood pressure estimation, or the like with high accuracy.

### DESCRIPTION OF THE DRAWINGS

FIG. 1 is a block diagram illustrating a biological signal detection device according to a first embodiment.

FIG. 2 is a circuit diagram illustrating a photo-reflector according to the first embodiment.

FIG. 3 is a block diagram illustrating an internal configuration of a reference pulse generation unit according to the first embodiment.

FIG. 4 is a flowchart illustrating operations of the reference pulse generation unit according to the first embodiment.

FIG. 5 is a block diagram illustrating an internal configuration of a reference voltage control unit according to the first embodiment.

FIGS. 6A and 6B are timing charts illustrating operations of a DUTY-variation amount detection unit according to the first embodiment.

FIG. 7 is a diagram illustrating a signal level of a biological detection signal by setting of a D/A converter according to the first embodiment.

FIG. 8 is a diagram illustrating a relationship between a variation amount detection signal and a biological detection signal according to the first embodiment.

FIG. 9 is a flowchart illustrating a reference voltage control in the biological signal detection device according to the first embodiment.

FIG. 10 is a diagram illustrating sensitivity adjustment of the biological detection signal according to the first embodiment.

FIG. 11 is a diagram illustrating correction processing of a reference voltage set value according to the first embodiment.

FIG. 12 is a flowchart illustrating the reference voltage control in a biological signal detection device according to a second embodiment.

FIG. 13 is a block diagram illustrating a biological signal detection device according to a third embodiment.

FIG. 14 is a block diagram illustrating an internal configuration of a reference voltage control unit according to the third embodiment.

FIG. 15 is a diagram illustrating correction processing of a reference voltage set value according to the third embodiment.

FIG. 16 is a flowchart illustrating the reference voltage control in a biological signal detection device according to a fourth embodiment.

FIG. 17 is a diagram illustrating an amplitude measurement of a photo-reflector output signal according to the fourth embodiment.

FIGS. 18A and 18B are diagrams illustrating an amplitude measurement of biological detection signal component according to the fourth embodiment.

FIG. 19 is a block diagram illustrating a biological signal detection device according to a fifth embodiment.

FIG. 20 is a block diagram illustrating an internal configuration of a reference voltage control unit according to the fifth embodiment.

FIG. 21 is a flowchart illustrating the reference voltage control in the biological signal detection device according to the fifth embodiment.

### DETAILED DESCRIPTION

According to one embodiment, there is provided a biological signal detection device capable of outputting a signal whose S/N ratio is large.

In general, according one embodiment, a biological signal detection device includes a reference pulse generation unit, a photo-reflector, a reference voltage control unit, a variation amount detection unit, and a biological signal generation unit. The reference pulse generation unit generates a reference pulse signal train having a frequency that is adjusted in response to a biological detection signal. The photo-reflector receives the reference pulse signal train, irradiates a living body, which is an object to be measured, with pulse light generated in response to the reference pulse signal train, and converts a light pulse train reflected from the living body into an electrical signal as a photo-reflector output signal train. The reference voltage control unit generates a reference voltage signal used for detection of a biological signal. The variation amount detection unit receives the photo-reflector output signal train and the reference voltage signal, performs difference processing between the reference voltage signal and the photo-reflector output signal train, and outputs a variation amount detection signal train. The biological signal generation unit receives the variation amount detection signal train and generates the biological detection signal.

In the following, embodiments will be described with reference to the drawings.

### First Embodiment

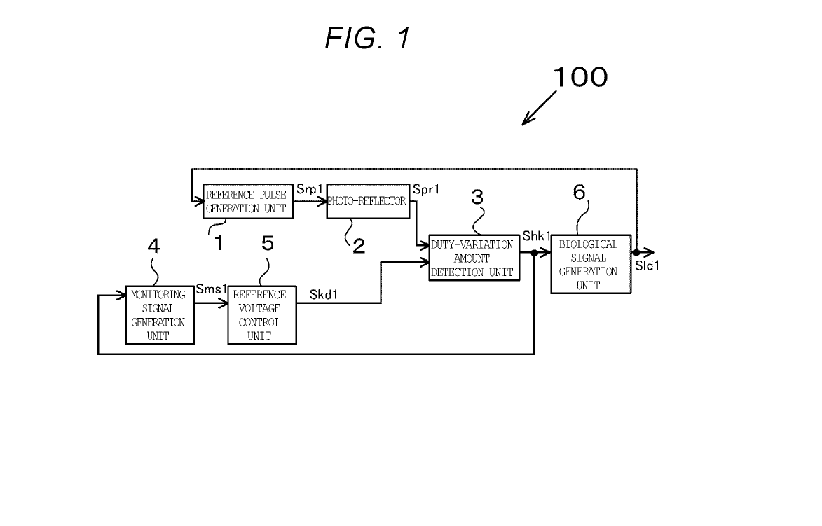

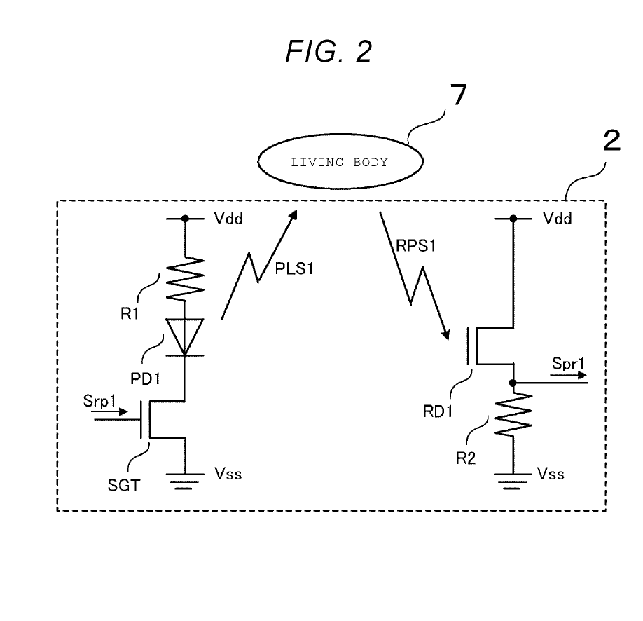

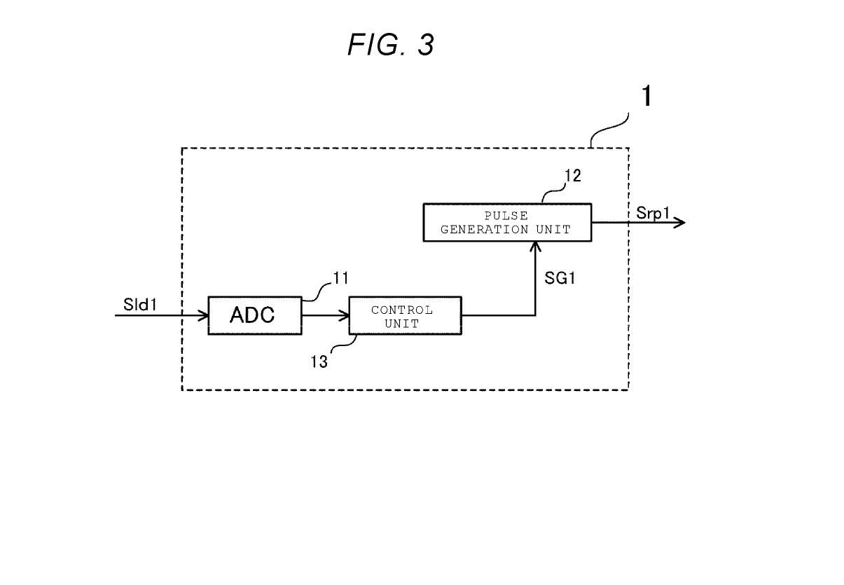

First, a biological signal detection device according to a first embodiment will be described with reference to the drawings. FIG. 1 is a block diagram illustrating a biological signal detection device. FIG. 2 is a circuit diagram illustrating a photo-reflector. FIG. 3 is a block diagram illustrating an internal configuration of a reference pulse generation unit. In the first embodiment, the biological signal is detected as a DUTY variation amount of a reference pulse train and a biological detection signal whose an S/N ratio is increased.

In the related art, a biological signal detection device has the following problems (illustration and details thereof are omitted).

1) An S/N ratio of a biological detection signal is very small.

2) The biological detection signal significantly varies due to an individual difference as an object to be measured.

3) Fine adjustment of sensitivity is difficult.

4) The power consumption of the biological signal detection device is large.

In contrast, according to the present embodiment, a biological signal detection device 100: 1) is able to increase the S/N ratio of the biological detection signal;

2) has a biological detection signal that does not vary by an individual difference as an object to be measured.

3) is capable of programming a wide and fine adjustment of sensitivity; and

4) is able to achieve low power consumption of the biological signal detection device.

A detailed description of each item is described below.

As illustrated in FIG. 1, the biological signal detection device 100 includes a reference pulse generation unit 1, a photo-reflector 2, a DUTY-variation amount detection unit 3, a monitoring signal generation unit 4, a reference voltage control unit 5, and a biological signal generation unit 6. The biological signal detection device 100 is applied to a pulse wave measurement of a living body.

The biological signal detection device 100 has a feedback loop structure. Specifically, in a first feedback loop, a biological detection signal Sld1 generated by the biological signal generation unit 6 is fed back to the reference pulse generation unit 1. In a second feedback loop, a variation amount detection signal Shk1 generated by the DUTY-variation amount detection unit 3 is fed back to the monitoring signal generation unit 4.

In the first feedback loop, the reference pulse generation unit 1 receives a biological detection signal Sld1, which is output from the biological signal generation unit 6, and generates a reference pulse signal train Srp1. The reference pulse generation unit 1 controls a frequency of the reference pulse signal Srp1 such that sensitivity of the biological detection signal Sld1 is an optimum value.

The photo-reflector 2 receives a reference pulse signal train Srp1, irradiates a living body, which is an object to be measured, with a pulse light train, and converts a pulse train reflected from the living body into a photo-reflector output signal train Spr1, which is an electrical signal, to be output.

The DUTY-variation amount detection unit 3 receives the photo-reflector output signal train Spr1 and a reference voltage signal Skd1 output from the reference voltage control unit 5, performs difference processing between the reference voltage signal Skd1 and the photo-reflector output signal train Spr1, and outputs a signal subjected to the difference processing as a variation amount detection signal train Shk1.

In the second feedback loop, the monitoring signal generation unit 4 receives the variation amount detection signal Shk1 and generates a monitoring signal Sms1. The monitoring signal generation unit 4 monitors states of respective circuits configuring the biological signal detection device 100 based on the variation amount detection signal train Shk1 and outputs a monitoring signal Sms1.

The reference voltage control unit 5 receives the monitoring signal Sms1 and generates a reference voltage signal Skd1. The reference voltage control unit 5 outputs the reference voltage signal Skd1 used for suitable detection of the DUTY variation amount without a difference of an object to be measured.

The biological signal generation unit 6 receives the variation amount detection signal train Shk1 and generates a biological detection signal Sld1 by converting the DUTY variation amount into a voltage level. The biological signal generation unit 6 includes an integration circuit. The integration circuit of the biological signal generation unit 6 has a cutoff frequency obtained by determining an effective frequency beyond which the Sld1 signal carries no biological information regarding the object to be measured. The integration circuit is provided so as to make it possible to increase the S/N ratio of the biological detection signal Sld1. For this reason, the biological signal detection device 100 is able to detect a signal with high accuracy even when being applied to, for example, finger plethysmogram of the finger belly of a living body which is object to be measured.

The biological detection signal Sld1 is not a feedback input to the reference pulse generation unit 1 at the start of a detection of a biological signal by the biological signal detection device 100. The variation amount detection signal train Shk1 is not a feedback input to the monitoring signal generation unit 4 at the start of detection. For this reason, the reference pulse signal Srp1 is output from the reference pulse generation unit 1 using a preset value. The monitoring signal Sms1 is also output from the monitoring signal generation unit 4 using the preset value.

As illustrated in FIG. 2, the photo-reflector 2 includes a light emitting element PD1, a resistor R1, a resistor R2, a light receiving element RD1, and a MOS transistor SGT. At a light emitting side of the photo-reflector 2, the resistor R1, the light emitting element PD1, and the MOS transistor SGT are provided. At a light receiving side of the photo-reflector 2, a light receiving element RD1 and a resistor R2 are provided.

One end of the resistor R1 is connected to a high potential side power supply Vdd. The anode of the light emitting element PD1 is connected to the other end of the resistor R1. The MOS transistor SGT is an enhanced (Etype) Nch MOS transistor. In the MOS transistor SGT, the drain is connected to the cathode of the light emitting element PD1, the source is connected to a low potential side power supply (ground potential) Vss, and the reference pulse signal Srp1 is input to the gate.

When the reference pulse signal Srp1 is in an enable state (for example, high level), the MOS transistor SGT is turned ON. When the MOS transistor SGT is turned ON, pulse light PLS1 is emitted from the light emitting element PD1 to a living body 7 which is an object to be measured.

In the present embodiment, the photo-reflector 2 is provided in the MOS transistor SGT. Only when the reference pulse signal Srp1 is in the enable state, the living body 7 irradiated with the pulse light PLS1. It is possible to achieve low power consumption of the biological signal detection device 100 compared to a biological signal generation unit in which the MOS transistor SGT is not provided.

The light receiving element RD1 is a photo-MOS transistor. One end of light receiving element RD1 is connected to the high potential side power supply Vdd. Reflected light pulse RPS1 reflected from the living body 7 is incident onto a control terminal and the photo-reflector output signal Spr1 is output from the other end side. Here, although a photo-MOS transistor is used in the light receiving element RD1, a bipolar transistor or a photodiode for light-receiving may also be used. One end of the resistor R2 is connected to the other end of the light receiving element RD1, and the other end of the resistor R2 is connected to the low potential side power supply (ground potential) Vss.

The living body 7 in FIG. 2 is assumed as a human body, but not necessarily limited thereto. For example, the biological signal detection device 100 may also be applied to animals including a pet in which blood flows, or the like.

As illustrated in FIG. 3, the reference pulse generation unit 1 includes an A/D converter 11, a pulse generation unit 12, and a control unit 13.

The A/D converter 11 receives the biological detection signal Sld1 which is an analog signal and is a feedback input, and performs analog/digital conversion processing. The control unit 13 receives a digital signal output from the A/D converter 11 and generates a control signal SG1. The pulse generation unit 12 generates the reference pulse signal train Srp1 having a fixed frequency based on the control signal SG1.

Here, the reference pulse signal train Srp1 is set in a frequency range of several kHz to several tens of kHz by taking AC characteristics of the photo-reflector 2 into account. For this reason, the photo-reflector output signal train Spr1 is a signal having a frequency in a range of several kHz to several tens of kHz.

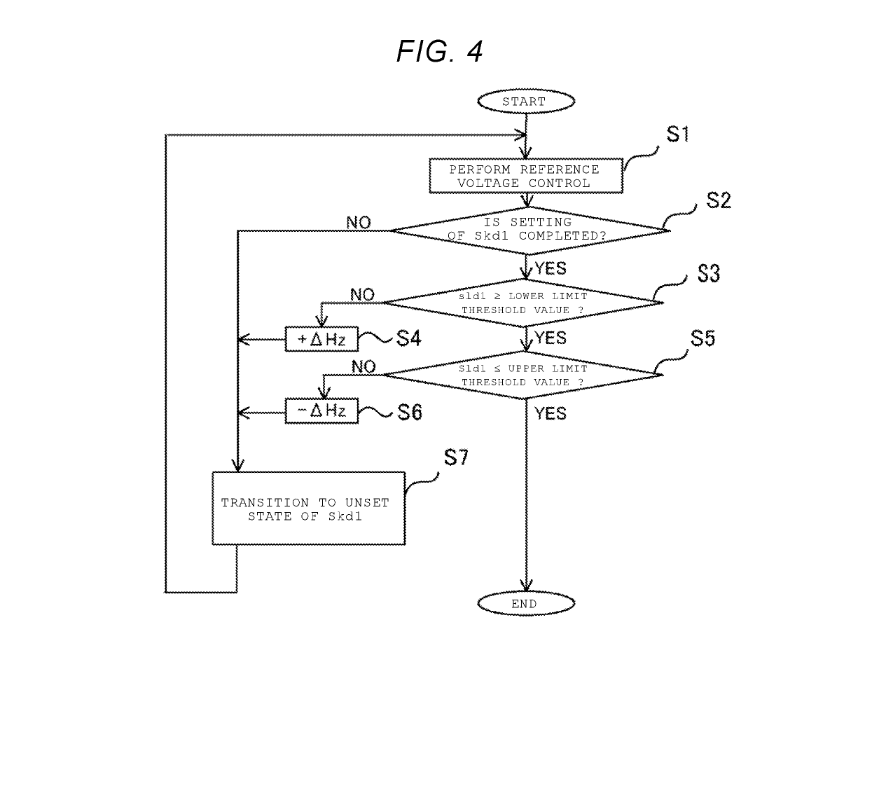

Referring to FIG. 4, the frequency control of the reference pulse generation unit 1 is next described. FIG. 4 is a flowchart illustrating operations of a reference pulse generation unit. Here, amplitude strength of the biological detection signal Sld1 is a value obtained by subtracting the minimum value of an output from the maximum value of the output. The maximum value and the minimum value of the output of biological detection signal Sld1 are determined by the flowchart.

As illustrated in FIG. 4, reference voltage control (D/A control) is performed (Step S1). Next, it is determined whether setting of the reference voltage output Skd1 (D/A setting) is completed (Step S2). When the setting is not completed, a flow of frequency control returns to the start of the flow through the transition to an unset state (Step S7).

When the setting is completed, it is determined whether the biological detection signal Sld1 is greater than or equal to a lower limit threshold value (Step S3). When the biological detection signal Sld1 is not greater than or equal to the lower limit threshold value, addition processing of ΔHz to the reference pulse frequency is executed (Step S4). After the addition processing, the flow of frequency control returns to the start of the flow through the transition to an unset state (Step S7).

When the biological detection signal Sld1 is greater than or equal to the lower limit threshold value, it is determined whether the biological detection signal Sld1 is less than or equal to an upper limit threshold value (Step S5). When the biological detection signal Sld1 is not less than or equal to an upper limit threshold value, subtraction processing of ΔHz from the reference pulse frequency is executed (Step S6). After the subtraction processing, the flow of frequency control returns to the start of the flow through the transition to an unset state (Step S7).

When the biological detection signal Sld1 is less than or equal to the upper limit threshold value, the reference pulse signal train Srp1 is suitably adjusted with respect to the measurement and output from the reference pulse generation unit 1.

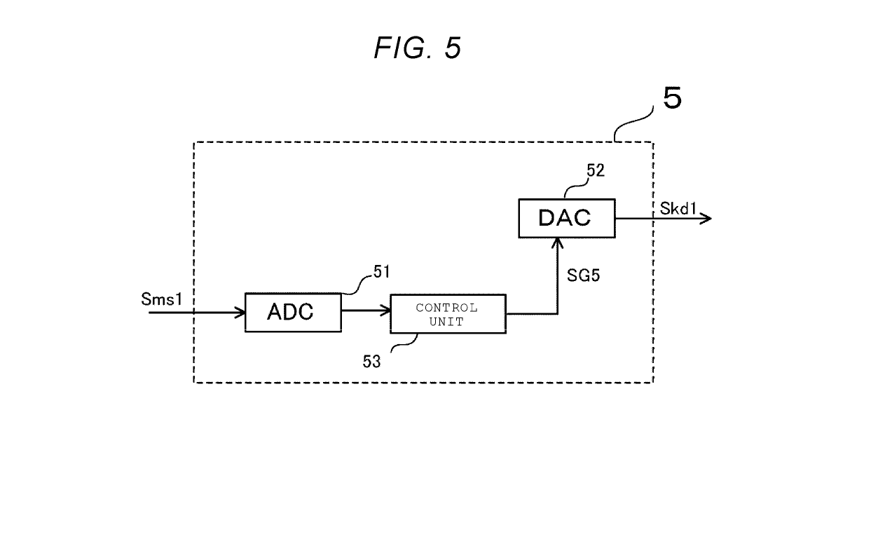

The reference voltage control unit 5 will be described with reference to FIG. 5. FIG. 5 is a block diagram illustrating an internal configuration of a reference voltage control unit.

As illustrated in FIG. 5, the reference voltage control unit 5 includes an A/D converter 51, a D/A converter 52, and a control unit 53.

The A/D converter 51 receives a monitoring signal Sms1 which is an analog signal output from the monitoring signal generation unit 4 and performs analog/digital conversion processing. The control unit 53 receives a digital signal output from the A/D converter 51 and generates a control signal SG5. The D/A converter 52 outputs a signal obtained by performing digital/analog conversion based on the control signal SG5 as the reference voltage signal Skd1.

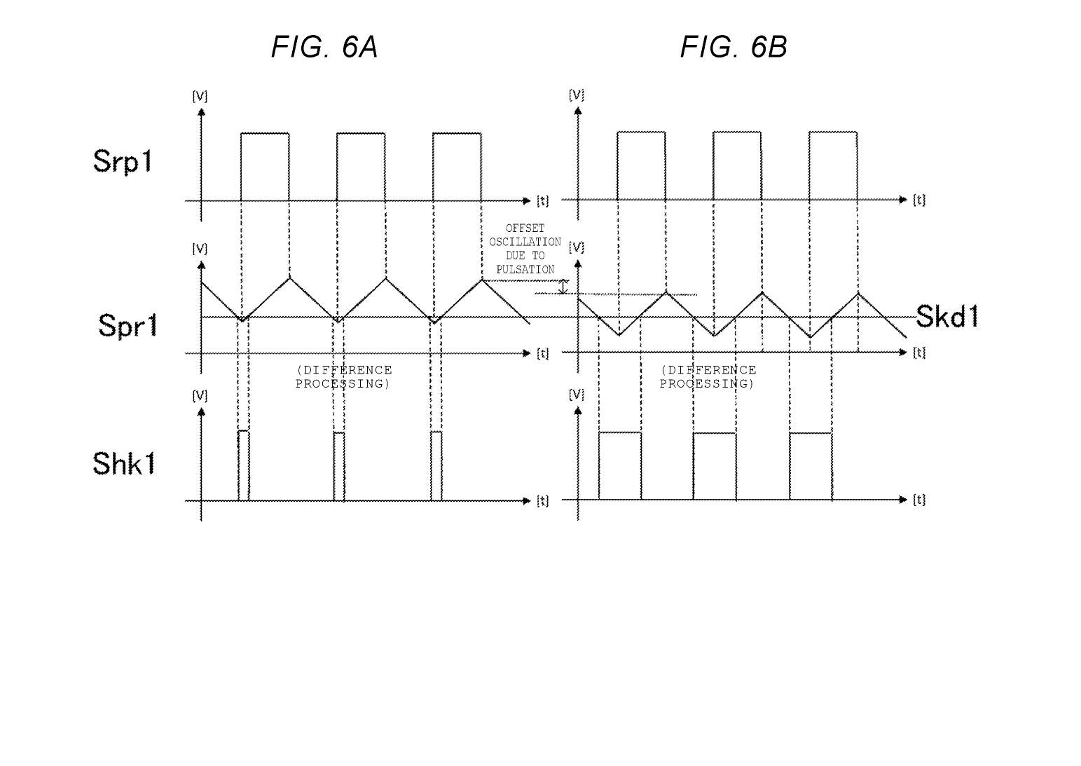

Operations of the DUTY-variation amount detection unit 3 will be described with reference to FIGS. 6A and 6B. FIGS. 6A and 6B are timing charts illustrating operations of a DUTY-variation amount detection unit. FIG. 6A illustrates a case where an offset of the photo-reflector output signal train Spr1 has a DC level that is comparatively large, and FIG. 6B illustrates a case where the offset of the photo-reflector output signal train Spr1 has a DC level that is comparatively small.

In the DUTY-variation amount detection unit 3, difference processing is performed between the reference voltage signal Skd1 and the photo-reflector output signal train Spr1 and a pulse width modulated signal train is obtained by performing the difference processing and output as a variation amount detection signal train Shk1. Here, the difference processing is subtraction processing of a value of the photo-reflector output signal train Spr1 from a value of the reference voltage signal Skd1, and a signal obtained by performing the subtraction processing is subjected to signal amplification.

As illustrated in FIG. 6A, when a DC offset of the photo-reflector output signal train Spr1 is comparatively large, a difference between the reference voltage signal Skd1 and minimum value of the photo-reflector output signal train Spr1 is small and a pulse width (high-level period) of the variation amount detection signal train Shk1 is small.

On the other hand, as illustrated in FIG. 6B, when the offset of the photo-reflector output signal train Spr1 is comparatively small, a difference between the reference voltage signal Skd1 and minimum value of the photo-reflector output signal train Spr1 is large and the pulse width (high-level period) of the variation amount detection signal train Shk1 is large.

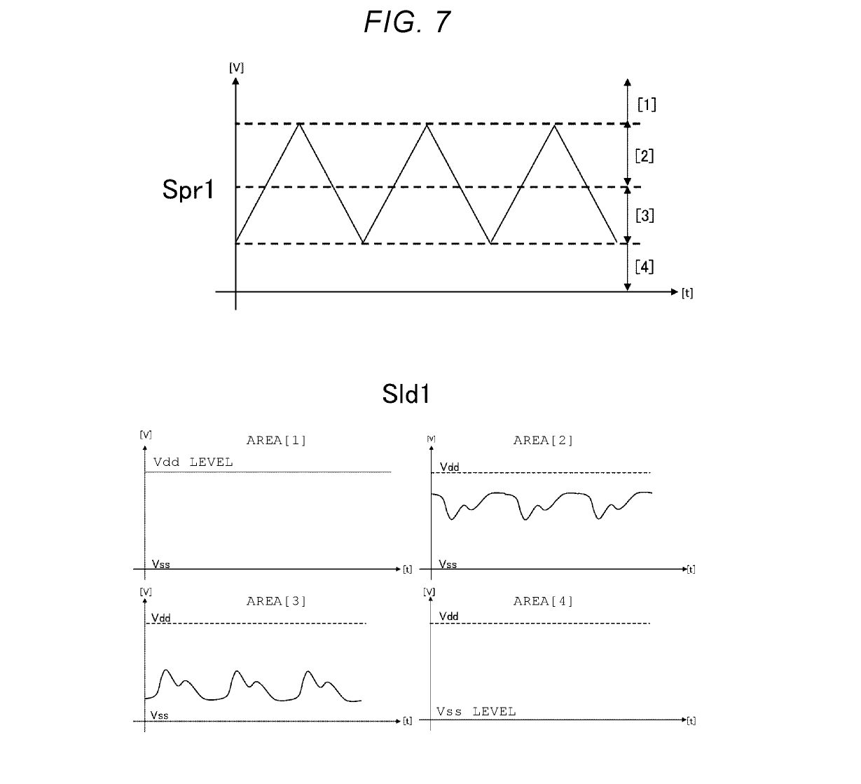

Referring to FIG. 7, the biological detection signal Sld1 is next described. FIG. 7 is a diagram illustrating a signal level of a biological detection signal by setting of a D/A converter. In the biological signal generation unit 6, an inversion type amplification circuit or a non-inversion type amplification circuit is arranged at a post-stage of the integration circuit. Here, the inversion type amplification circuit is used in the area [2] and the non-inversion type amplification circuit is used in the area [3].

As illustrated in FIG. 7, when the reference voltage signal Skd1 is in the area [1], the biological detection signal Sld1 has a Vdd level (a fixed high level).

When the reference voltage signal Skd1 is in the area [2], the biological detection signal Sld1 is an inverted signal by the inversion type amplification circuit.

When the reference voltage signal Skd1 is in the area [3], the biological detection signal Sld1 is a non-inverted signal by the non-inversion type amplification circuit.

When the reference voltage signal Skd1 is in the area [4], the biological detection signal Sld1 is a Vss level (a fixed low level).

In the present embodiment, the reference voltage signal Skd1 is set to the area [3] by repeating processing. However, an offset value of a waveform of the photo-reflector output signal train Spr1 dynamically varies due to pulsation of the biological detection signal Sld1. For this reason, a circuit parameter or an input frequency of the photo-reflector 2 is set to a suitable value such that a variation value is sufficiently small with respect to the area [3].

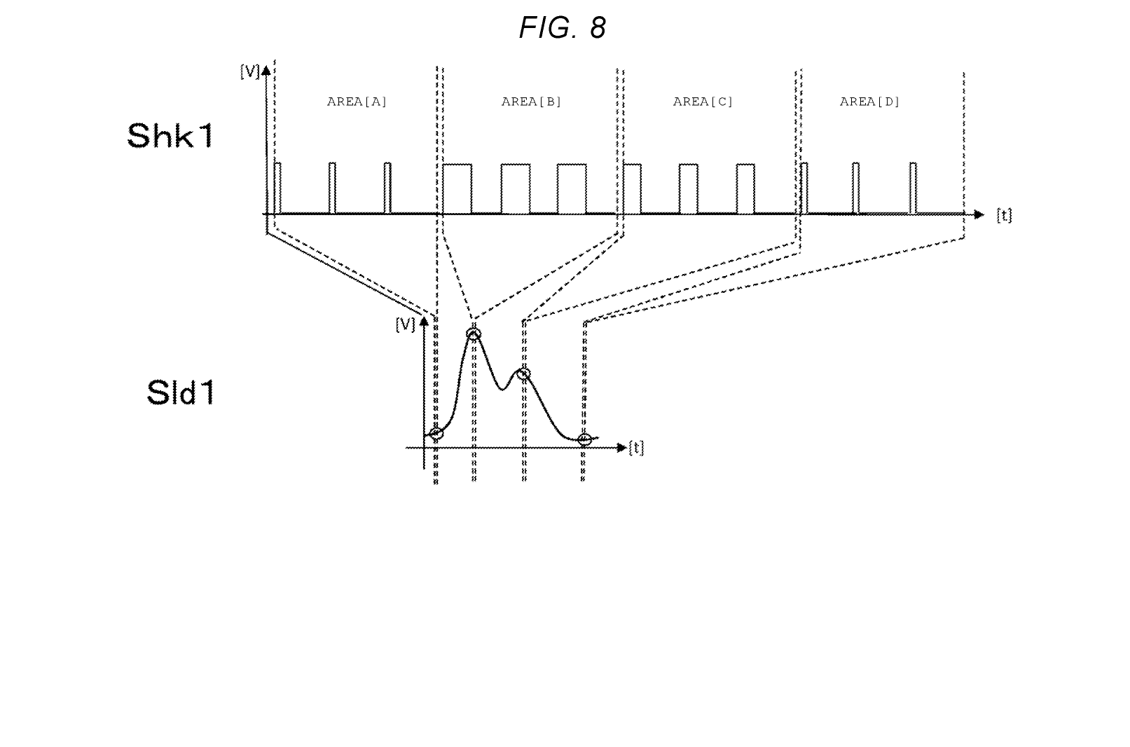

Referring to FIG. 8, a relationship between the variation amount detection signal train Shk1 and the biological detection signal Sld1 is next described. FIG. 8 is a diagram illustrating a relationship between a variation amount detection signal train and a biological detection signal.

As illustrated in FIG. 8, here, the frequency of the variation amount detection signal train Shk1 is in, for example, a range of several kHz to several tens of kHz, and the frequency of the biological detection signal Sld1 is approximately 1 Hz. When a pulse width (high-level period) of the variation amount detection signal train Shk1 is narrow, a signal level of biological detection signal Sld1 is a small value. The signal level of the biological detection signal Sld1 gradually becomes a larger value as the pulse width (high-level period) of the variation amount detection signal train Shk1 is increases.

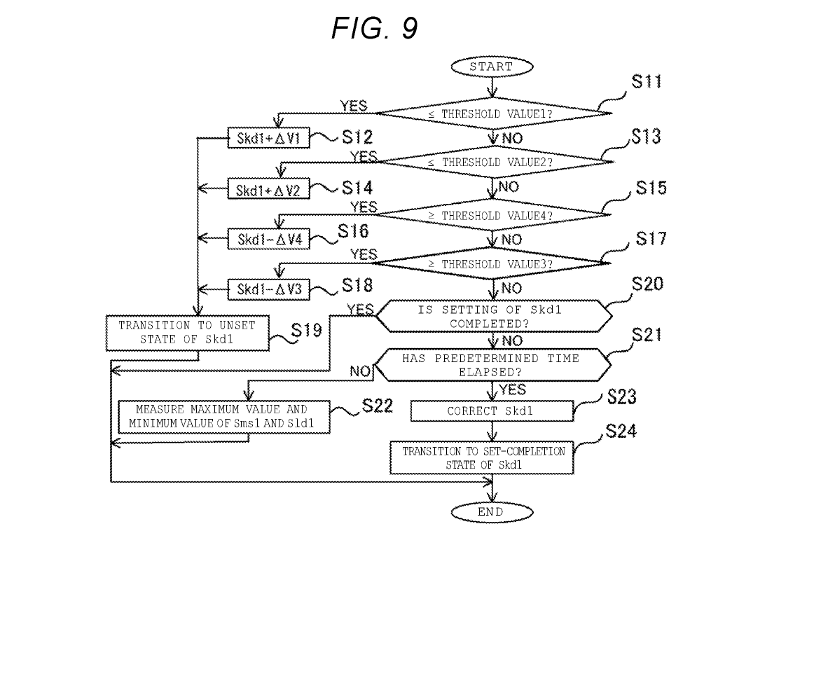

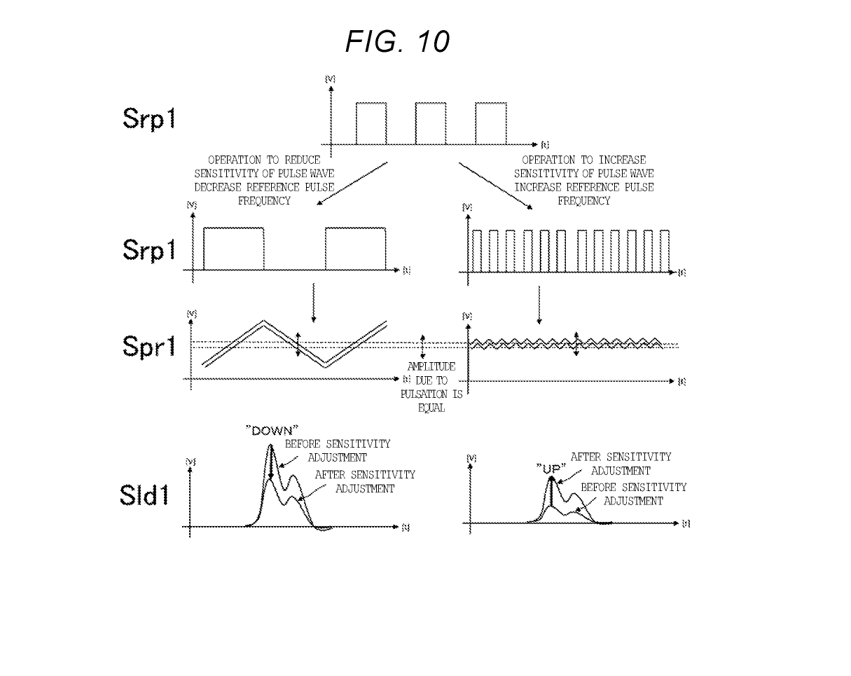

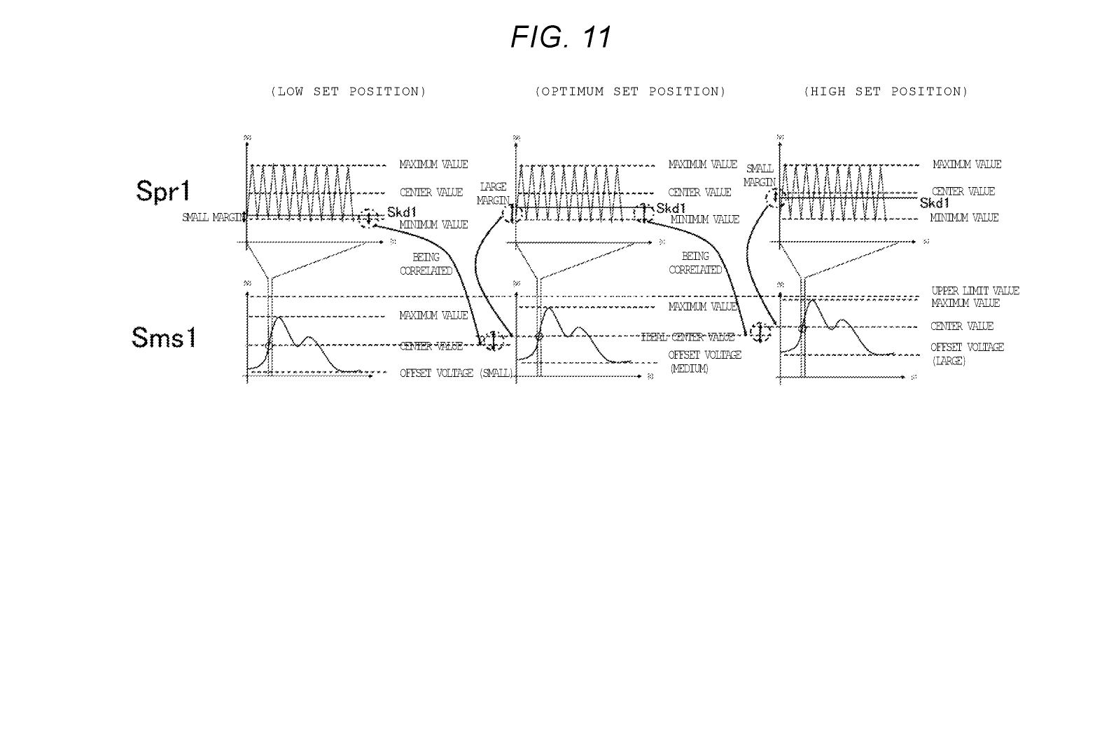

Next, referring to FIGS. 9 to 11, the reference voltage control (D/A control processing) of the biological signal detection device 100 is described, where FIG. 9 is a flowchart illustrating the reference voltage control, FIG. 10 is a diagram illustrating sensitivity adjustment of the biological detection signal, and FIG. 11 is a diagram illustrating correction processing of a reference voltage set value.

In the reference voltage control (D/A control processing) of the biological signal detection device 100 according to the first embodiment, settings are made according to the following.

a) Correcting the reference voltage signal Skd1 is performed by setting the threshold value of the monitoring signal Sms1 to satisfy the relationship threshold value 1<threshold value 2<Sms1<threshold value 3<threshold value 4.

b) The reference voltage signal Skd1 is set to the area [3] in FIG. 7 by repeatedly executing the flow of FIG. 9.

c) In voltage setting, ΔV1>ΔV2 and −ΔV3>−ΔV4 are established.

d) A predetermined time is set to be greater than or equal to one period of the biological detection signal Sld1.

As illustrated in FIG. 9, it is determined whether the monitoring signal Sms1 is greater than or equal to the threshold value 1 (Step S11). If the monitoring signal Sms1 is not greater than the threshold value 1, ΔV1 is added to the reference voltage signal Skd1 which is an output of the D/A converter 52 (Step S12). Then, a flow of reference voltage control transitions to an unset state of Skd1 and the timer counter is cleared to 0 (zero) (Step S19).

If the monitoring signal Sms1 is greater than the threshold value 1, it is determined whether the monitoring signal Sms1 is greater than the threshold value 2 (Step S13). If the monitoring signal Sms1 is not greater than the threshold value 2, ΔV2 is added to the reference voltage signal Skd1 which is an output of the D/A converter 52 (Step S14). Then, a flow of reference voltage control transitions to an unset state of Skd1 and the timer counter is cleared to 0 (zero) (Step S19).

If the monitoring signal Sms1 is greater than the threshold value 2, it is determined whether the monitoring signal Sms1 is greater than or equal to the threshold value 4 (Step S15). If the monitoring signal Sms1 is greater than or equal to the threshold value 4, ΔV4 is subtracted from the reference voltage signal Skd1 which is an output of the D/A converter 52 (Step S16). Then, a flow of reference voltage control transitions to an unset state of Skd1 and the timer counter is cleared to 0 (zero) (Step S19).

If the monitoring signal Sms1 is not greater than or equal to the threshold value 4, it is determined whether the monitoring signal Sms1 is greater than or equal to the threshold value 3 (Step S17). If the monitoring signal Sms1 is greater than or equal to the threshold value 3, ΔV3 is subtracted from the reference voltage signal Skd1 which is an output of the D/A converter 52 (Step S18). Then, a flow of reference voltage control transitions to an unset state of Skd1 and the timer counter is cleared to 0 (zero) (Step S19).

If the monitoring signal Sms1 is not greater than or equal to the threshold value 3, it is determined whether a flow of reference voltage control is to be transitioned to a set-completion state of the Skd1 (Step S20). When at the set-completion state of the Skd1, processing of the reference voltage control is ended. When a set completion flag is not 1, it is determined whether a predetermined time has elapsed (Step S21).

If the predetermined time has not elapsed (timer counter<predetermined time), processing is ended after measurement of the maximum value and the minimum value of the monitoring signal Sms1 and the biological detection signal Sld1 (Step S22). If the timer counter is greater than or equal to the predetermined time, correcting of the Skd1 is performed (Step S23). After the correction of the Skd1, a set-completion flag of the D/A converter 52 is set to 1 and processing is completed.

As illustrated in FIG. 10, in the sensitivity adjustment of biological detection signal Sld1, when a frequency of the reference pulse signal train Srp1 is too large, an operation of decreasing the frequency of the reference pulse signal train Srp1 so as to reduce the sensitivity of the pulse wave is executed. When the frequency of the reference pulse signal Srp1 is too small, an operation of increasing the frequency of the reference pulse signal Srp1 so as to increase the sensitivity of the pulse wave is executed.

The frequency is turned UP or DOWN to thereby automatically adjust sensitivity of the biological detection signal Sld1 while maintaining a duty ratio (a proportion of high-level period/low-level period) of the reference pulse signal train Srp1 constant.

As illustrated in FIG. 11, if a set position is low or when the set position is high, correction processing sets the preset voltage value of the reference voltage signal Skd1 to an optimum set position using a correction value.

With Vrc1 as the ideal center value of the monitoring signal Sms1, Vc1 as the center value of the monitoring signal Sms1, Vps as the amplitude value of the photo-reflector output signal train Spr1, and Vu as the upper limit value of the monitoring signal Sms1, the correction value Vc is set according to Equation (1).

Vc={(Vrc1−Vc1)/Vu}×(Vps/2) Equation (1).

With Vhm as the value of the reference voltage signal Skd1 before the correction, the value Vhg of the reference voltage signal Skd1 after the correction is according to Equation (2).

Vhg=Vhm+Vc Equation (2).

Here, DUTY of the variation amount detection signal Shk1 is preferably defined when the monitoring signal Sms1 is within respective levels of the ideal center value and the upper limit value illustrated in FIG. 11. The ideal center value and the upper limit value can be calculated by a theoretical calculation when respective rectangular pulses are input to the integration circuit of the biological signal generation unit 6. As the amplitude of the photo-reflector output signal train Spr1, values obtained by measuring amplitudes of the photo-reflector output signal train Spr1 in advance and stored in a parameter table may be preferably used.

By signal processing (see FIG. 9) in the biological signal detection device 100, as long as Skd1 is within the threshold values and the signal level of the monitoring signal Sms1 does not exceed the upper limit value, the signal level of the reference voltage signal Skd1 stays fixed, i.e., no signal processing according to FIG. 9 is performed. When the processing described above is executed, it is done at intervals shorter than a sampling frequency of the biological signal to thereby make it possible to keep a suitable state of the biological detection signal Sld1.

As described above, in the biological signal detection device according to the present embodiment, the reference pulse generation unit 1, the photo-reflector 2, the DUTY-variation amount detection unit 3, the monitoring signal generation unit 4, the reference voltage control unit 5, and the biological signal generation unit 6 are provided. The reference pulse generation unit 1 receives the biological detection signal Sld1 which is feedback-input and generates the reference pulse signal Srp1. The photo-reflector 2 receives the reference pulse signal train Srp1 and outputs reflected light pulse train RPS1 reflected from the living body 7 as the photo-reflector output signal train Spr1.

The DUTY-variation amount detection unit 3 receives the photo-reflector output signal train Spr1 and the reference voltage signal Skd1, performs difference processing between the reference voltage signal Skd1 and the photo-reflector output signal train Spr1, and outputs a signal subjected to the difference processing as the variation amount detection signal Shk1. The monitoring signal generation unit 4 receives the variation amount detection signal train Shk1 which is a feedback input and generates the monitoring signal Sms1. The reference voltage control unit 5 receives the monitoring signal Sms1 and generates the reference voltage signal Skd1. The biological signal generation unit 6 receives the variation amount detection signal train Shk1 and generates the biological detection signal Sld1. The biological signal generation unit 6 includes the integration circuit. The reference voltage signal Skd1 is corrected to be in the optimum set value. A frequency of the reference pulse signal train Srp1 is adjusted. In the photo-reflector 2, the MOS transistor SGT which is turned ON only when the reference pulse signal train Srp1 is in an enable state.

With this arrangement, it is possible to increase the S/N ratio of the biological detection signal Sld1. The pulse wave sensitivity of the living body 7 may be made available in a state that is matched with each person. Additionally, it is possible to adjust sensitivity of the biological detection signal Sld1 and to achieve low power consumption of the biological signal detection device 100.

In the present embodiment, the pulse wave measurement of the living body 7 as the object to be measured is conducted with high sensitivity using the biological signal detection device 100. Alternatively, the biological signal detection device 100 may also be applied to a vital sensing technology such as the stress measurement, SpO2 (percutaneous arterial blood oxygen saturation) measurement, blood pressure estimation, or the like using the pulse wave.

In the present embodiment, the variation amount detection signal train Shk1 is a feedback input to the monitoring signal generation unit 4 but is not necessarily limited thereto. For example, the variation amount detection signal train Shk1 and the photo-reflector output signal train Spr1 maybe a feedback input to the monitoring signal generation unit 4. In this case, the monitoring signal Sms1 is generated by the monitoring signal generation unit 4 based on the variation amount detection signal train Shk1 and the photo-reflector output signal train Spr1.

### Second Embodiment

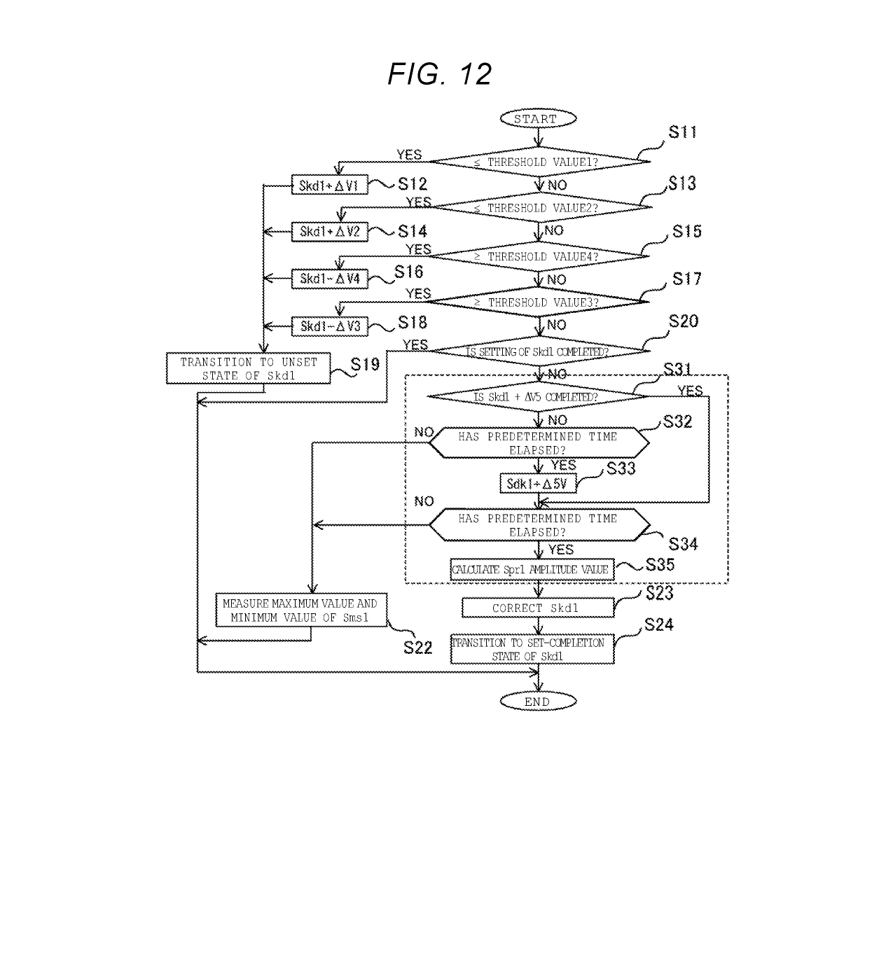

A biological signal detection device according to a second embodiment will be described with reference to the drawings. FIG. 12 is a flowchart illustrating operations for reference voltage control in a biological signal detection device. In the second embodiment, the biological signal is detected as a DUTY variation amount of a reference pulse train and a biological detection signal whose the S/N ratio is increased is output.

Although the present embodiment has the same configuration as that of the first embodiment, the reference voltage control and the monitoring signal of the second embodiment are different from those of the first embodiment. Steps included in an area surrounded by a dotted line in FIG. 12 are added.

In the following, the same configuration portions of as those of the first embodiment are assigned the same reference symbols and descriptions thereof will be omitted and different portions will be described.

In the reference voltage control (D/A control processing) of the biological signal detection device according to the second embodiment, a) setting of a threshold value, b) setting to be the area [3] in FIG. 7, c) voltage setting, and d) the predetermined time are the same as those of the biological signal detection device 100 according to the first embodiment.

As illustrated in FIG. 12, when setting of the output value Skd1 of the D/A converter is not completed in Step S20, it is determined whether ΔV5 has been already added to the D/A converter 52 (Step S31). If ΔV5 has been already added, Steps S32 and S33 are skipped. If ΔV5 is not yet added, it is determined whether a value of a timer counter is greater than a predetermined time (Step S32).

If the value is not greater than the predetermined time, processing of the reference voltage control is ended through the measurement (Step S22) of the maximum value and the minimum value of the monitoring signal Sms1 and the biological detection signal Sld1. If the value is greater than the predetermined time, ΔV5 is added to the reference voltage signal Skd1 which is an output of the D/A converter 52 (Step S33).

Next, it is determined whether the value of the timer counter is greater than a predetermined time (Step S34). If the value is not greater than the predetermined time, processing of the reference voltage control is ended through the measurement (Step S22) of the maximum value and the minimum value of the monitoring signal Sms1 and the biological detection signal Sld1. If the value is greater than the predetermined time, an output amplitude value of the photo-reflector 2 is calculated (Step S35). After this, descriptions of the same Steps as those of the first embodiment will be omitted.

Here, the center value of the monitoring signal Sms1 after the output value Skd1 of the D/A converter is varied to ΔV5 is set as Vc1a, the center value of the monitoring signal Sms1 before the output value Skd1 of the D/A converter is varied to ΔV5 is set as Vc1b and a relationship among the amplitude value Vps of the photo-reflector output signal Spr1, the upper limit value Vu, and ΔV5 is set to establish Vps={(2×Vu)/Vc1a−Vc1b)}×ΔV5 . . . Equation (3).

By setting of Equation (3), it is possible to autonomously use an optimum amplitude value of the photo-reflector output signal train Spr1 without using the frequency of the reference pulse signal train Srp1 and the parameter table of amplitude values of the photo-reflector output signal train Spr1 as in the first embodiment.

As described above, in the biological signal detection device according to the second embodiment, determining whether addition has been already added to signal processing according to the first embodiment, a first determination whether a value of the timer counter is greater than a predetermined time, a ΔV5 addition, and second determination whether a value of the timer counter is greater than a predetermined time, and calculation of an output amplitude value are added.

For this reason, it is possible to autonomously use an optimum amplitude value of the photo-reflector output signal train Spr1 in addition to effects according to the first embodiment.

### Third Embodiment

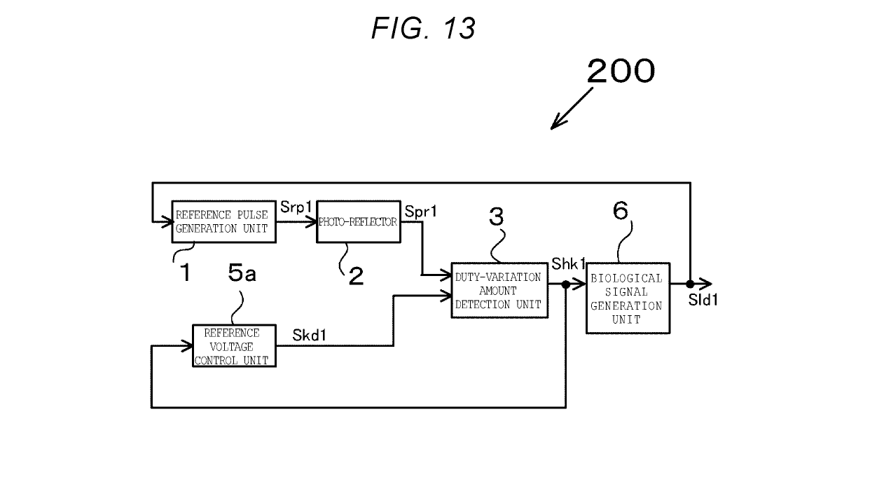

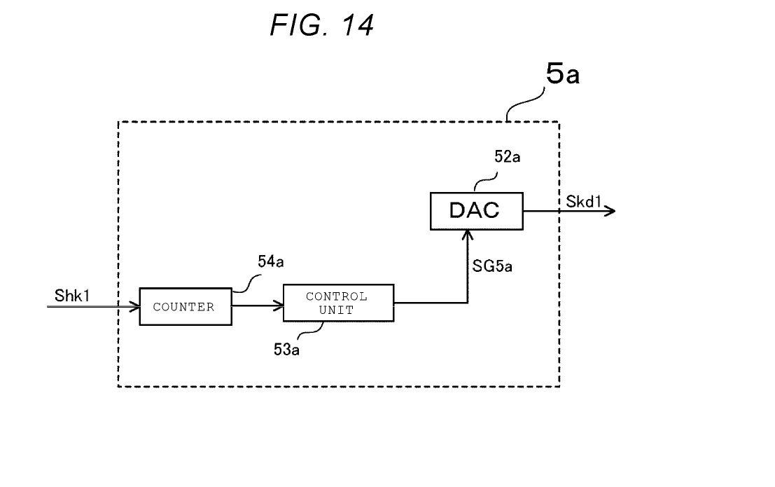

A biological signal detection device according to a third embodiment will be described with reference to the drawings. FIG. 13 is a block diagram illustrating a biological signal detection device. FIG. 14 is a block diagram illustrating an internal configuration of a reference voltage control unit. In the present embodiment, the biological signal is detected as a DUTY variation amount of a reference pulse and a biological detection signal whose S/N ratio is increased is output.

In the following, the same configuration portions of as those of the first embodiment are assigned the same reference symbols and descriptions thereof will be omitted and different portions will be described.

As illustrated in FIG. 13, a biological signal detection device 200 includes the reference pulse generation unit 1, the photo-reflector 2, the DUTY-variation amount detection unit 3, a reference voltage control unit 5a, and the biological signal generation unit 6. The biological signal detection device 200 is applied to a pulse wave measurement of a living body. In the biological signal detection device 200 according to the present embodiment, the monitoring signal generation unit 4 of the biological signal detection device 100 according to the first embodiment is omitted.

The biological signal detection device 200 has a feedback loop structure. Specifically, the biological detection signal Sld1 generated by the biological signal generation unit 6 is a feedback input to the reference pulse generation unit 1. The variation amount detection signal Shk1 generated by the DUTY-variation amount detection unit 3 is a feedback input to the reference voltage control unit 5a.

As illustrated in FIG. 14, the reference voltage control unit 5a receives the variation amount detection signal train Shk1, which is a feedback input, as a monitoring signal and generates the reference voltage signal Skd1. The reference voltage control unit 5a includes a D/A converter 52a, a control unit 53a, and a counter 54a.

A counter 54a receives the variation amount detection signal train Shk1, which is a feedback input, as the monitoring signal and measures a pulse width of the variation amount detection signal train Shk1 using the counter. A control unit 53a receives a count signal of the counter 54a and generates a control signal SG5a. The D/A converter 52a generates a signal obtained by performing a digital/analog conversion as the reference voltage signal Skd1 based on the control signal SG5a.

In signal processing of the biological signal detection device according to the present embodiment, the same Steps (see FIG. 9) as those in the signal processing according to the first embodiment are performed. a) setting of a threshold value, b) setting to the area [3] in FIG. 7, c) voltage setting, and d) a predetermined time are the same as those in the biological signal detection device 100 according to the first embodiment.

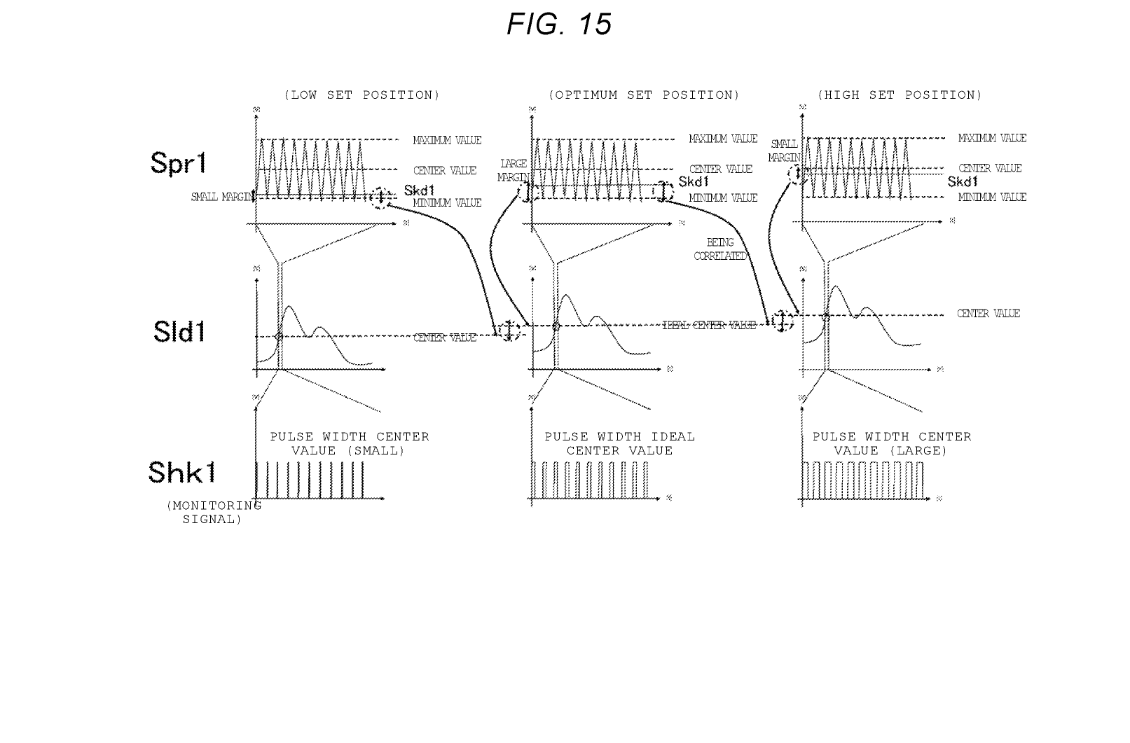

Next, correction processing of the Skd1 in the signal processing of the biological signal detection device will be described with reference to FIG. 15. FIG. 15 is a diagram illustrating correction processing of a reference voltage set value Skd1.

As illustrated in FIG. 15, in the correction processing of a set voltage value of the reference voltage signal Skd1, when a set position is low or when the set position is high, the set voltage value is set to the optimum set position using a correction value.

The upper limit value Vu is set as a value of DUTY 50% (high-level period). The relationship between an ideal center value Vrc11 of the variation amount detection signal Shk1 which is a monitoring signal, a center value Vc11 of the variation amount detection signal Shk1 which is the monitoring signal, the amplitude value Vps of the photo-reflector output signal Spr1, the upper limit value Vu, and the correction value Vc is set to establish Vc={(Vrc11−Vc11)/Vu}×(Vps/2) . . . Equation (4).

The relationship between a value Vhm of the reference voltage signal Skd1 before the correction and a value Vhg of the reference voltage signal Skd1 after the correction is the same as Equation (2) described in the first embodiment, and the value Vhg of the reference voltage signal Skd1 after the correction is obtained by adding the correction value Vc to the value Vhm of the reference voltage signal Skd1 before the correction.

Here, values determined in advance, for amplitude values of the photo-reflector output signal train Spr1 preferably, are preferably stored in a parameter table and programmed.

As described above, in the biological signal detection device according to the present embodiment, the reference pulse generation unit 1, the photo-reflector 2, the DUTY-variation amount detection unit 3, the reference voltage control unit 5a, and the biological signal generation unit 6 are provided. The reference voltage control unit 5a receives the variation amount detection signal train Shk1, which is a feedback input, as the monitoring signal and generates the reference voltage signal Skd1.

For this reason, in addition to the effects of the first embodiment, it is possible to simplify constituent elements compared to the biological signal detection device 100 according to the first embodiment.

### Fourth Embodiment

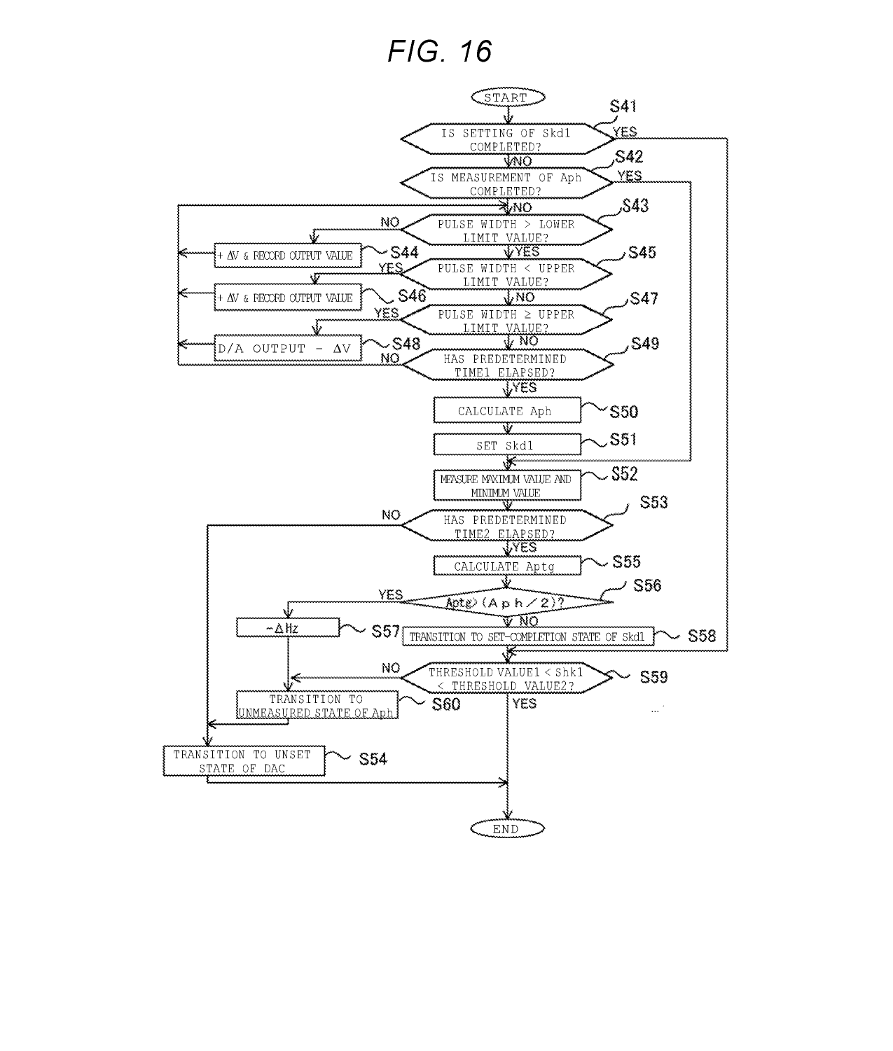

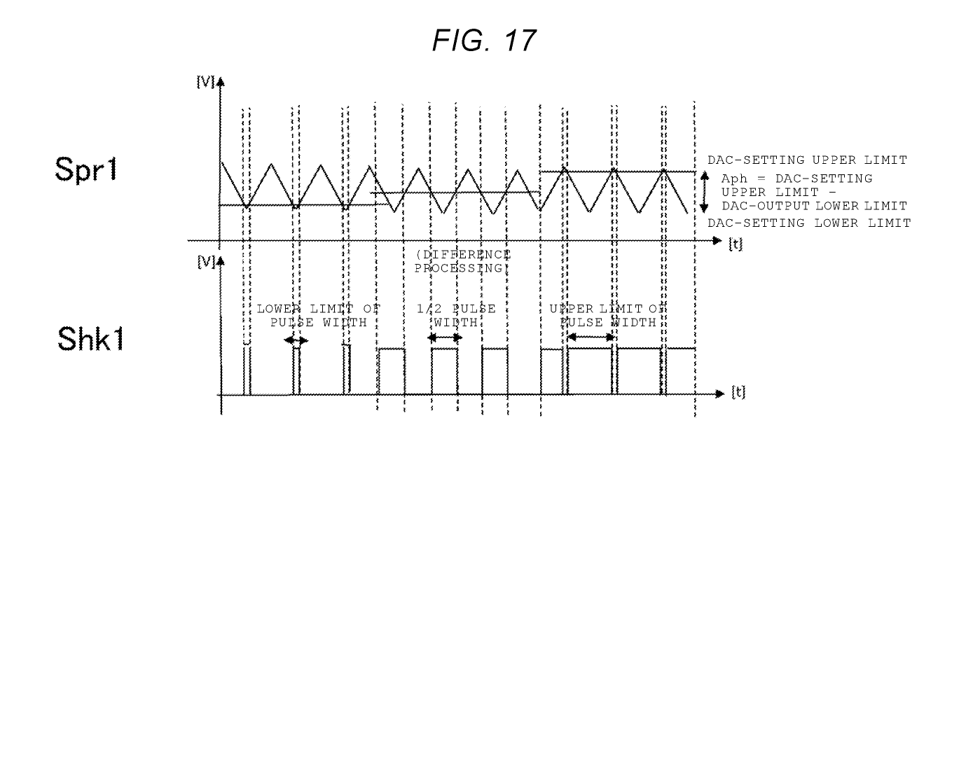

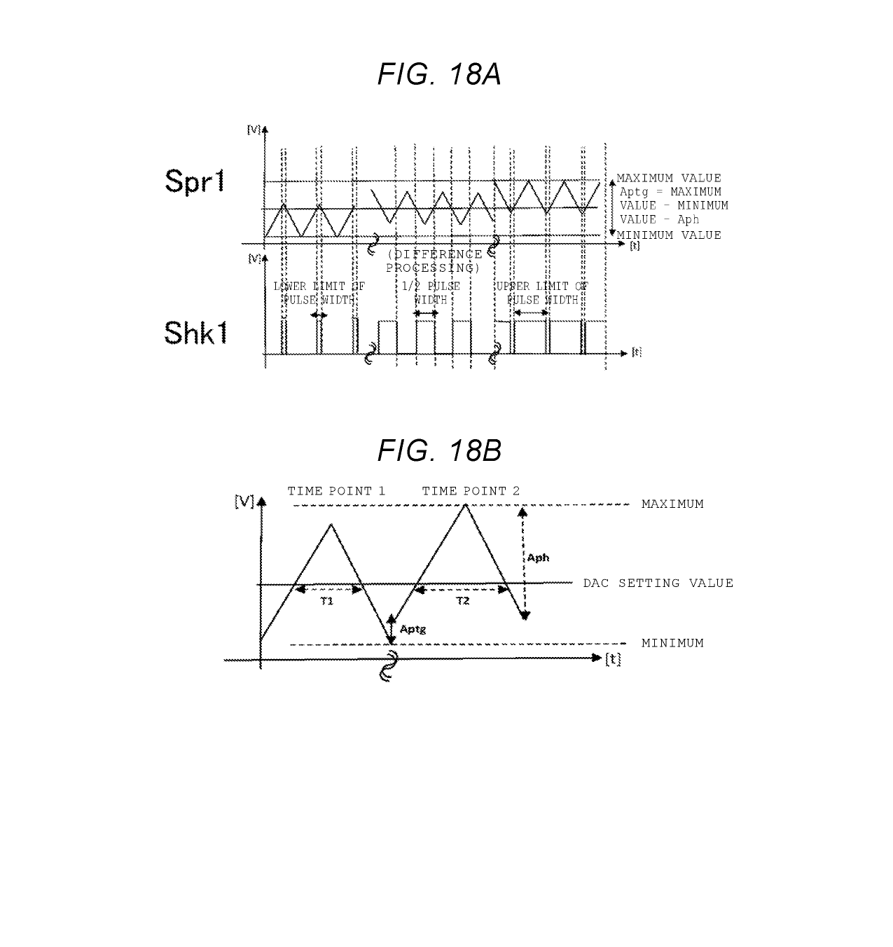

Next, a biological signal detection device according to a fourth embodiment will be described with reference to the drawings. FIG. 16 is a flowchart illustrating operations for reference voltage control in a biological signal detection device. FIG. 17 is a diagram illustrating an amplitude measurement of a photo-reflector output signal. FIGS. 18A and 18B are diagrams illustrating an amplitude measurement of a biological detection signal component. In the present embodiment, the biological signal is detected as a DUTY variation amount of a reference pulse and a biological detection signal whose S/N ratio is increased is output.

In the present embodiment, the biological signal detection device has the same configuration as the biological signal detection device 200 according to the third embodiment.

In the following, the same configuration portions of as those of the first embodiment and third embodiment are assigned the same reference symbols and descriptions thereof will be omitted and different portions will be described.

In the reference voltage control (D/A control processing) according to the fourth embodiment, the threshold value is a threshold value with respect to the pulse width (see FIGS. 6A and 6B) of the DUTY-variation amount detection unit 3 and setting is made to be threshold value 1<threshold value 2. A predetermined time 1 corresponds to the time greater than or equal to one period of the variation amount detection signal Shk1 as the monitoring signal. A predetermined time 2 corresponds to the time greater than or equal to one period of the biological detection signal Sld1.

As illustrated in FIG. 16, it is determined whether setting of the D/A converter 52a (see FIG. 14) is completed (Step S41). When the setting is completed, a flow of the reference voltage control proceeds to Step S59. When the setting is not completed, it is determined whether an Aph measurement is completed (Step S42). When the Aph measurement is completed, a flow of the reference voltage control proceeds to Step S52.

Here, the Aph is the amplitude of a reference pulse component of the photo-reflector output signal Spr1 and is obtained by subtracting a set lower limit value from a set upper limit value of the D/A converter 52a.

The Aph measurement is performed as illustrated in FIG. 17. Search of the photo-reflector output signal train Spr1 is performed for a period greater than or equal to one period of the reference pulse signal train Srp1. With respect to the photo-reflector output signal train Spr1, the variation amount detection signal train Shk1, which is the monitoring signal, is measured by varying an output Skd1 of the D/A converter 52a from the set upper limit value to the set lower limit value. A pulse width of the variation amount detection signal train Shk1, which is the monitoring signal, is subjected to pulse width measurement and the pulse width is measured by the counter 54a (see FIG. 14) to thereby control the pulse width such that the output Skd1 of the D/A converter 52a is in the area [3] in FIG. 7.

If the Aph measurement is not completed, it is determined whether the pulse width of the variation amount detection signal train Shk1, which is the monitoring signal, is greater than or equal to the lower limit value (Step S43). When the pulse width of the variation amount detection signal Shk1 is not greater than or equal to the lower limit value, the flow of the reference voltage control returns to Step S43 through addition of ΔV to the output Skd1 of the D/A converter 52a and storing of the output value (Step S44).

If the pulse width of the variation amount detection signal train Shk1 is greater than or equal to the lower limit value, it is determined whether the pulse width of the variation amount detection signal Shk1, which is the monitoring signal, is less than or equal to the upper limit value (Step S45). If the pulse width of the variation amount detection signal Shk1 is less than or equal to the upper limit value, the flow of the reference voltage control returns to Step S43 through addition of ΔV to the output Skd1 of the D/A converter 52a and storing of the output value (Step S46).

If the pulse width of the variation amount detection signal Shk1 is not less than or equal to the upper limit value, it is determined whether the pulse width of the variation amount detection signal train Shk1, which is the monitoring signal, is greater than or equal to the upper limit value (Step S47). When the pulse width is greater than or equal to the upper limit value, the flow of the reference voltage control returns to Step S43 through subtraction of ΔV from the output Skd1 of the D/A converter 52a (Step S48).

If the pulse width is not greater than or equal to the upper limit value, it is determined whether the measurement time of the counter 54a is greater than the predetermined time (Step S49). If the pulse width is not greater than the predetermined time 1, the flow of the reference voltage control returns to Step S43.

If the pulse width is greater than or equal to the predetermined time 1, an Aph calculation illustrated in FIG. 17 is executed (Step S50). Next, setting of the D/A converter 52a is made to (set upper limit of DAC+set lower limit of DAC)/2 (Step S51). Subsequently, the maximum value and the minimum value of the biological detection signal Sld1 are measured (Step S52).

It is determined whether the measurement time of the counter 54a is greater than the predetermined time 2 (Step S53). If the predetermined time 2 has not elapsed, processing of the reference voltage control is ended through transition to an unset state of DAC (Step S54).

If the predetermined time 2 has elapsed, an Aptg calculation is executed based on the maximum value, the minimum value, and the Aph (Step S55).

As illustrated in FIG. 18A, the Aptg is the amplitude of the pulsation component of the photo-reflector output signal train Spr1 and is obtained by subtracting the minimum value of the photo-reflector output signal train Spr1 and the amplitude Aph (see FIG. 17) of the reference pulse component of the photo-reflector output signal train Spr1 from the maximum value of the photo-reflector output signal train Spr1. Search of the photo-reflector output signal train Spr1 is performed for a period greater than or equal to one period of the biological detection signal Sld1.

The time T1 illustrated in FIG. 18B is an interval between adjacent intersection points of the photo-reflector output signal train Spr1 and the DAC setting value at time point 1. Time T2 is an interval between adjacent intersection points of the photo-reflector output signal train Spr1 and the DAC setting value at time point 2. When a pulse period of the photo-reflector output signal Spr1 is set as T, the relationship between the Aptg, Aph, T2, T3, T1, and T is set to establish Aptg={(T2−T1)/T}×Aph . . . Equation (5). With Vma as the maximum value of the photo-reflector output signal Spr1, Vmi as the minimum value of the photo-reflector output signal Spr1, the DAC setting value Vds according to Equation (6) is

Vds={(Vma+Vmi)/2}−(Aph/4) Equation (6).

Next, it is determined whether the Aptg, which is the amplitude of the pulsation component of the photo-reflector output signal Spr1, is greater than (Aph/2) (Step S56). If the Aptg is greater than the (Aph/2), the processing of the reference voltage control is ended through subtraction of the frequency of the reference pulse signal Srp1 by ΔHz (Step S57), transition to an unmeasured state of Aph (Step S60), and transition to an unset state of DAC (Step S54).

If the Aptg is not greater than the (Aph/2), the DAC setting value represented in Equation (6) is calculated (Step S58).

Subsequently, it is determined whether when the variation amount detection signal train Shk1, which is the monitoring signal, is greater than the threshold value 1 and less than the threshold value 2 (Step S59). If the variation amount detection signal is greater than the threshold value 1 and not less than the threshold value 2, the processing of the reference voltage control is ended through transition to an unmeasured state of Aph (Step S60) and transition to an unset state of DAC (Step S54). If the variation amount detection signal is greater than the threshold value 1 and less than the threshold value 2, the processing is ended.

As described above, in the biological signal detection device according to the fourth embodiment, the Aptg which is the amplitude of the pulsation component of the photo-reflector output signal Spr1 and the DAC setting value are calculated so as to execute signal processing by the biological signal detection device.

For this reason, the circuit is simplified and adjustment sensitivity is improved in addition to the effects of the first embodiment.

### Fifth Embodiment

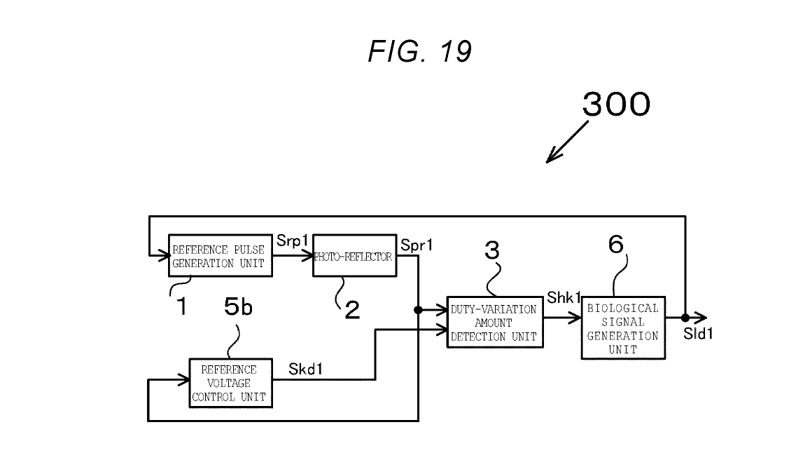

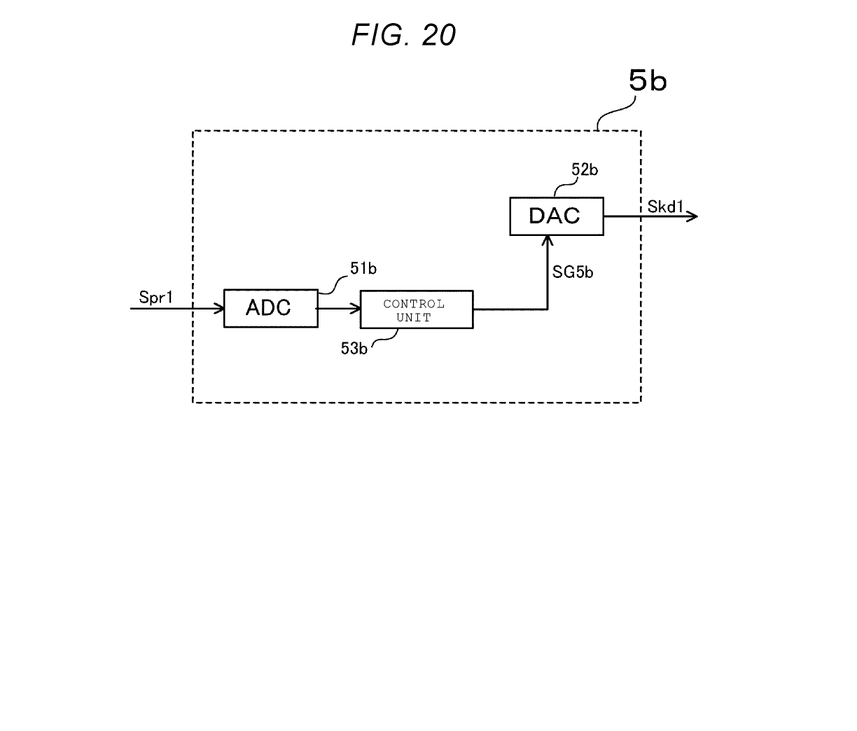

Next, a biological signal detection device according to a fifth embodiment will be described with reference to the drawings. FIG. 19 is a block diagram illustrating biological signal detection device. FIG. 20 is a block diagram illustrating an internal configuration of a reference voltage control unit. In the present embodiment, the biological signal is detected as a DUTY variation amount of a reference pulse train and a biological detection signal whose S/N ratio is increased is output.

In the following, the same configuration portions of as those of the first embodiment and fourth embodiment are assigned the same reference symbols and descriptions thereof will be omitted and different portions will be described.

As illustrated in FIG. 19, a biological signal detection device 300 includes the reference pulse generation unit 1, the photo-reflector 2, the DUTY-variation amount detection unit 3, a reference voltage control unit 5b, and the biological signal generation unit 6. The biological signal detection device 300 is applied to a pulse wave measurement of a living body. In the biological signal detection device 300 according to the fifth embodiment, the monitoring signal generation unit 4 of the biological signal detection device 100 according to the first embodiment is omitted.

The biological signal detection device 300 has a feedback loop structure. Specifically, the biological detection signal Sld1 generated by the biological signal generation unit 6 is a feedback input to the reference pulse generation unit 1. The photo-reflector output signal Spr1 generated by the photo-reflector 2 is a feedback input to the reference voltage control unit 5b.

As illustrated in FIG. 20, the reference voltage control unit 5b receives the photo-reflector output signal train Spr1, which is a feedback input, as a monitoring signal and generates the reference voltage signal Skd1. The reference voltage control unit 5b includes an A/D converter 51b, a D/A converter 52b, and a control unit 53b.

The A/D converter 51b receives the photo-reflector output signal train Spr1, which is a feedback input, as the monitoring signal and performs analog/digital conversion processing on the photo-reflector output signal train Spr1. The control unit 53b receives a signal which is subjected to the analog/digital conversion processing by the A/D converter 51b and generates a control signal SG5b. The D/A converter 52b generates the signal subjected to the digital/analog conversion processing as the reference voltage signal Skd1 based on the control signal SG5b.

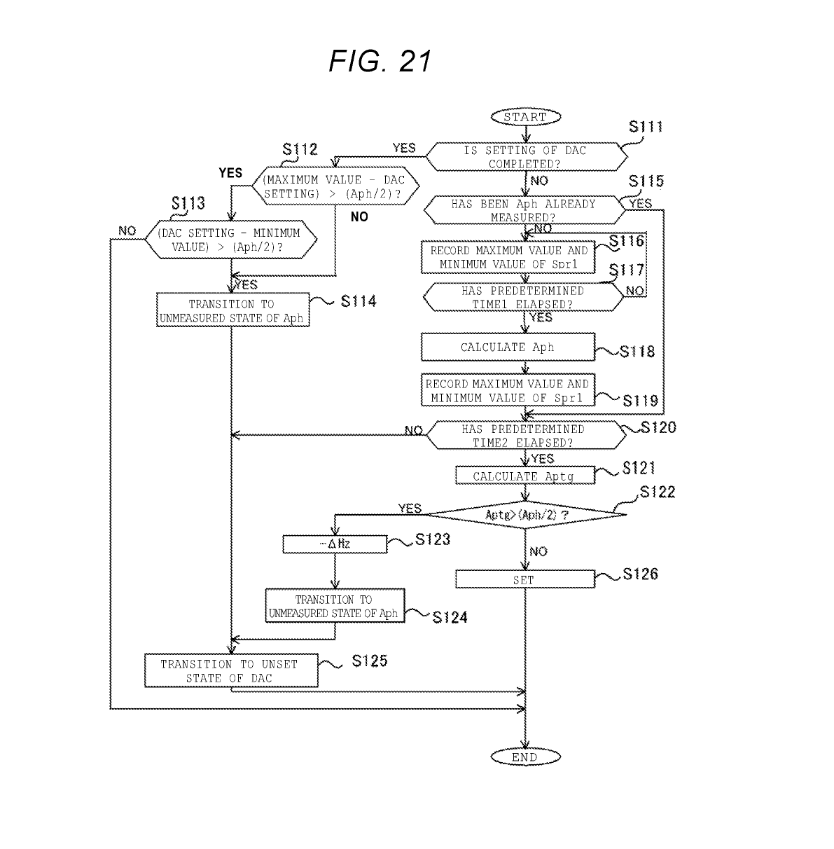

Next, reference voltage control (D/A control processing) of the biological signal detection device 300 will be described with reference to FIG. 21. FIG. 21 is a flowchart illustrating operations for reference voltage control in a biological signal detection device. In signal processing of the biological signal detection device 300 according to the present embodiment, settings are made as in the following.

In the signal processing of the present embodiment, the amplitude of the reference pulse component of the photo-reflector output signal Spr1 is set as Aph and the amplitude of the pulsation component of the photo-reflector output signal Spr1 is set as Aptg. A predetermined time 1 corresponds to the time greater than or equal to one period of the variation amount detection signal Shk1 as the monitoring signal. A predetermined time 2 corresponds to the time greater than or equal to one period of the biological detection signal Sld1. The DAC setting value is set to establish Equation (6) (see fourth embodiment).

As illustrated in FIG. 21, it is determined whether DAC setting is completed (Step S111). If the DAC setting is completed, it is determined whether the (maximum value−DAC setting) is greater than the (Aph/2) (Step S112). If the (maximum value−DAC setting) is not greater than the (Aph/2), a flow of reference voltage control proceeds to transition to an unset state of Aph (Step S114).

If the (maximum value−DAC setting) is greater than the (Aph/2), it is determined whether the (DAC setting−minimum value) is greater than the (Aph/2) (Step S113). If the (DAC setting−minimum value) is not greater than the (Aph/2), processing of the reference voltage control is ended.

If the (DAC setting−minimum value) is greater than the (Aph/2), processing of the reference voltage control is ended through transition to an unmeasured state of Aph (Step S114) and transition to an unset state of DAC (Step S125).

If the DAC setting is not completed, it is determined whether the Aph has been already measured (Step S115). If the Aph has been already measured, the flow of reference voltage control is skipped to Step S120. If the Aph is not measured, the maximum value and the minimum value of the photo-reflector output signal train Spr1 as the monitoring signal are recorded (Step S116).

Next, it is determined whether the predetermined time has elapsed using a timer counter (Step S117). The predetermined time 1 corresponds to a period greater than or equal to one period of the reference pulse signal Srp1. Here, the timer counter (not illustrated) is provided in the reference voltage control unit 5b.

If the predetermined time 1 has not elapsed, the flow of reference voltage control returns to Step S116. If the predetermined time 1 has elapsed, the Aph is calculated by subtraction processing (maximum value−minimum value) of the photo-reflector output signal Spr1 as the monitoring signal (Step S118).

Subsequently, the maximum value and the minimum value of the photo-reflector output signal Spr1 as the monitoring signal are recorded (Step S119).

Next, it is determined whether the predetermined time 2 has elapsed (Step S120). If the predetermined time 2 has not elapsed, the processing of reference voltage control is ended through transition to an unset state of DAC (Step S125).

If the predetermined time 2 has elapsed, the Aptg is calculated based on (maximum value−minimum value−Aph) of the photo-reflector output signal Spr1 as the monitoring signal (Step S121).

Subsequently, it is determined whether the Aptg is greater than the (Aph/2) (Step S122). If the Aptg is greater than the (Aph/2), the processing of reference voltage control is ended through subtraction of the frequency of the reference pulse signal Srp1 by ΔHz (Step S123), transition to an unmeasured state of Aph (Step S124), and transition to an unset state of DAC (Step S125).

If the Aptg is not greater than the (Aph/2), a DAC setting value is calculated (see Equation (6) of fourth embodiment) and set to a DAC output (Step S126). By doing as described above, the processing of reference voltage control is completed.

As described above, in the biological signal detection device according to the present embodiment, the reference pulse generation unit 1, the photo-reflector 2, the DUTY-variation amount detection unit 3, a reference voltage control unit 5b, and the biological signal generation unit 6 are provided. The photo-reflector output signal train Spr1 generated by the photo-reflector 2 is feedback-input to the reference voltage control unit 5b as the monitoring signal.

For this reason, in addition to the effects of the first embodiment, it is possible to simplify constituent elements compared to those of the biological signal detection device 100 according to the first embodiment.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein maybe made without departing from the spirit of the inventions. The accompanying claims and their equivalents are intended to cover such forms or modifications as would fall within the scope and spirit of the inventions.

Configurations described in the following Attachments may be possible.

What is claimed is:

1. A biological signal detection device, comprising:
a reference pulse generation unit that generates a reference pulse signal train having a frequency that is adjusted in response to a biological detection signal;
a photo-reflector that receives the reference pulse signal train, irradiates a living body, which is an object to be measured, with pulse light generated in response to the reference pulse signal train, and converts a light pulse train reflected from the living body into an electrical signal as a photo-reflector output signal train;
a reference voltage control unit that generates a reference voltage signal used for detection of a biological signal;
a variation amount detection unit that receives the photo-reflector output signal train and the reference voltage signal, performs difference processing between the reference voltage signal and the photo-reflector output signal train, and outputs a variation amount detection signal train; and
a biological signal generation unit that receives the variation amount detection signal train and generates the biological detection signal.

2. The device according to claim 1, further comprising:
a monitoring signal generation unit that receives the variation amount detection signal train, generates a monitoring signal controlling the reference voltage control unit, and outputs the monitoring signal to the reference voltage control unit.

3. The device according to claim 1,
wherein the reference voltage control unit receives the variation amount detection signal train and generates the reference voltage signal using the variation amount detection signal train as a monitoring signal controlling the reference voltage control unit.

4. The device according to claim 3,
wherein the reference voltage control unit includes a counter, a first control unit, and a D/A converter,
wherein the counter receives the variation amount detection signal train as a monitoring signal, and measures a pulse width of the variation amount detection signal train,
wherein the first control unit receives a count signal of the counter and generates a first control signal, and
wherein the D/A converter generates a signal obtained by performing a digital/analog conversion as the reference voltage signal based on the first control signal.

5. The device according to claim 1,
wherein the reference voltage control unit receives the photo-reflector output signal train and generates the reference voltage signal using the photo-reflector output signal train as a monitoring signal controlling the reference voltage control unit.

6. The device according to claim 1,
wherein the photo-reflector includes a light emitting element and a MOS transistor at a side from which the pulse light is emitted to the living body,
wherein the reference pulse signal train is input to a gate of the MOS transistor and the MOS transistor is turned ON when the reference pulse signal train is in an enable state, and
wherein the light emitting element irradiates the living body with the pulse light when the MOS transistor is turned ON.

7. The device according to claim 1,
wherein a frequency of the reference pulse signal train is controlled to adjust sensitivity of the biological detection signal.

8. The device according to claim 1,
wherein the reference pulse generation unit includes an A/D converter, a second control unit, and a pulse generation unit,
wherein the A/D converter receives the biological detection signal and generates a signal obtained by performing an analog/digital conversion, and
wherein the second control unit receives the signal obtained by performing the analog/digital conversion and generates the reference pulse signal as a reference pulse signal train based on a second control signal of the second control unit.

9. The device according to claim 1,
wherein the biological signal generation unit includes an integration circuit, and
wherein the integration circuit has a cutoff frequency obtained by determining an effective frequency beyond which the biological detection signal carries no biological information regarding the object to be measured.

10. A biological signal detection device comprising:
a biological signal generation unit that generates a biological detection signal in response to a variation amount detection signal train;
a reference pulse generation unit that generates a reference pulse signal train having a variable frequency that is adjusted in response to the biological detection signal;
a photo-reflector that receives the reference pulse signal train, irradiates living tissue with pulse light generated in response to the reference pulse signal train, and converts a light pulse train, reflected from the living tissue, into an electrical signal as a photo-reflector output signal train;
a reference voltage control unit that generates a reference voltage signal for comparison with the photo-reflector output signal train; and
a duty-variation amount detection unit that receives the photo-reflector output signal train and the reference voltage signal, performs a comparison between the reference voltage signal and the photo-reflector output signal train, and outputs a pulse width modulated signal train as the variation amount detection signal train.

11. The biological signal detection device according to claim 10, wherein the biological signal generation unit generates a voltage signal as the biological detection signal train by creating a voltage that is proportional to a width of each pulse of the pulse width modulated signal train.

12. The biological signal detection device according to claim 10, wherein the reference pulse generation unit includes:
an A/D converter that converts the biological detection signal to a digital signal; and
a pulse generation unit that generates the reference pulse signal train based on the digital signal, wherein a frequency of the reference pulse signal train is set in response to the digital signal by comparing the frequency against a lower limit threshold value and an upper limit threshold value and adjusting the frequency to be between the lower and upper threshold values.

13. The biological signal detection device according to claim 10, wherein the photo-reflector includes:
a light emitting element that receives the reference pulse signal train and generates the pulse light train; and
a light receiving element that receives the reflected light pulse trains and generates the photo-reflector output signal train.

14. The biological signal detection device according to claim 10, wherein the monitoring signal generation unit generates a voltage proportional to pulse widths of the variation amount detection signal train.

15. The biological signal detection device according to claim 10, wherein a duty-variation amount detection unit generates the pulse width modulated signal train by determining, for each signal in the photo-reflector output signal train, a length of time that each signal of the photo-reflector output signal train is below the reference voltage signal.

16. The biological signal detection device according to claim 10,
wherein the reference voltage control unit generates the reference voltage in response to a voltage control signal;
further comprising a monitoring signal generation unit that receives the variation amount detection signal and generates the voltage control signal.

17. The biological signal detection device according to claim 16, wherein the reference voltage control unit generates the reference voltage based on the voltage control signal by adjusting the voltage control signal to be greater than the first and second threshold values and less than the third and fourth threshold values.

18. The biological signal detection device according to claim 16, wherein the reference voltage control unit generates the reference voltage based on the voltage control signal by adjusting the voltage control signal to be greater than the first and second threshold values and less than the third and fourth threshold values and adding a fifth value to the adjusted voltage control signal.

19. The biological signal detection device according to claim 10, wherein the reference voltage control unit generates the reference voltage in response to the variation amount detection signal.

20. The biological signal detection device according to claim 10, wherein the reference voltage control unit generates the reference voltage in response to the photo-reflector output signal train.
